(19) United States
(12) Patent Application Publication
(10) Pub. No.: US 2002/0058312 A1
(43) Pub. Date: May 16, 2002

(54) Expression cassetts and methods for delivery of animal vaccines

(76) Inventors: Benjamin P. All, Johnston, IA (US); John A. Howard, College Station, TX (US)

Correspondence Address: ZARLEY MCKEE THOMTE VOORHEES & SEASE PLC, SUITE 3200, 801 GRAND AVENUE, DES MOINES, IA, 50309-2721, US

(21) Appl. No.: 09/797,097

(22) Filed: Mar. 1, 2001

Related U.S. Application Data
(63) Continuation of application No. 08/529,006, filed on Sep. 15, 1995.

(51) Int. Cl. G01N 33/53; C12P 21/04; A01H 5/00

(52) U.S. Cl. 435/71.1; 800/288

(57) ABSTRACT

The present invention provides an expression cassette for expressing vaccine antigens in a plant cell. The expression cassette includes a DNA sequence which encodes for at least one vaccine antigen which is operably linked to transcriptional and translational control regions functional in the plant cell. The vaccine antigens of the invention are useful for protection of an animal against mucosal diseases such as Transmissible Gastroenteritis Virus (TGEV) and rotavirus. The invention also provides a transgenic plant and transgenic plant seed which has been stably transformed to express a vaccine antigen which is included in an expression cassette of the invention. The transformed plant and plant cells may be from monocot or dicot plants and include, for example, corn, soybeans, sunflower, canola or alfalfa.

The transgenic plants and plant seeds of the invention may be used as a feed composition for animals. Alternatively, the transgenic plant and plant seeds of the invention may provide an immunogenic composition for protecting animals against mucosal diseases after oral administration.

### BACKGROUND OF THE INVENTION

Diseases of the mucosal tissue, such as those affecting the enteric system, the respiratory tract, urogenital tract and mammary glands are of significant economic impact in domestic animals. These diseases include, for example, the Bovine Respiratory Disease Complex (BRDC), bovine and porcine rotavirus and coronavirus, bacterial pathogens such as Pasteurella spp. and Haemophilus spp., mastitis in dairy cattle and abortion-inducing pathogens such as Leptospira spp. and Campylobacter fetus. Mucosal immunity is of prime importance in protection against these diseases. Secretory IgA (SIgA) is the predominant immunoglobulin relevant to the prevention of infection of mucosal surfaces. The main protective function of SIgA antibodies is the “immune exclusion” of bacterial and viral pathogens, bacterial toxins and other antigens. The immune response generated at the surface of one mucosal tissue site can be disseminated to other mucosal sites due to the migration of lymphocytes to other mucosal tissue, thus providing immunity at all mucosal tissue sites.

Once mucosal immunity is established in an animal it can be advantageously transferred to the offspring. Immunity in neonates may be passively acquired through colostrum and/or milk. This has been referred to as lactogenic immunity and is an efficient way to protect animals during early life. SIgA is the major immunoglobulin in milk and is most efficiently induced by mucosal immunization.

It is now widely recognized that mucosal immunity is generally best induced by direct immunization of the mucosal tissue. In order to enhance efficacy against mucosal diseases, vaccines should stimulate the mucosal system and generate an SIgA immune response. One way of achieving this goal is by administering the vaccine orally and targeting the mucosal tissue lining the gastrointestinal tract. Studies support the potential of inducing SIgA antibody formation and immune protection in “distant” extra-intestinal mucosal sites after oral vaccination. Activated lymphocytes from the gut can disseminate immunity to other mucosal and glandular tissues. Therefore, oral vaccines can protect against infections at sites remote from the antigenic stimulation, for example in the respiratory and urogenital tracts.

Oral mucosal vaccines have the potential of providing a more user-friendly and economical approach to vaccination than current parenteral vaccines. They would be easier to administer since minimal supervision by medically trained personnel or equipment would be required. Oral vaccination also has the potential to achieve wide distribution, which is particularly suited for immunization of large populations of animals.

The principal challenge of delivering an oral vaccine is to be able to present adequate amounts of the antigen to the intestinal mucosa where it can stimulate the gut mucosal system to generate SIgA and induce lasting immunity. There are three types of vaccines which can be given orally and which have been or are currently being developed: 1) live vaccines; 2) vectored vaccines; and 3) sub-unit vaccines. A fourth type, inactivated vaccines, typically require parenteral administration.

However, there are disadvantages in using currently available oral vaccines for animals. The disadvantages for live vaccines or vectored vaccines are that vaccine strains can revert toward virulence, some live vaccine strains are not recommended for use in pregnant animals, they are difficult to generate and they can be contaminated during preparation. Sub-unit vaccines can be difficult to produce recombinantly if they are glycosylated, can be difficult to purify from transformed cells, can be inherently unstable, and can be expensive because large repeated oral doses can be required in order to elicit mucosal immunity. There is a need to develop a less expensive system for producing and delivering target vaccine antigens to animals.

Transgenic plants have been used to produce heterologous or foreign proteins. Some examples to date are the production of interferon in tobacco (Goodman et al., 1987), enkephalins in tobacco, Brassica napus and Ababidopsis thaliana (Vandekerchove et al., 1989), human serum albumin in tobacco and potato (Sijmons et al., 1990) antibodies in tobacco (Hiatt et al., 1990) and hepatitis B antigen (Mason et al., 1992). The use of transgenic plants for producing vaccines has been suggested, however, there has been no showing in these references of expression in plants at levels sufficient to protect animals against disease or that oral immunization with the plant would be effective to protect animals, particularly domestic animals, against disease.

Thus, there is a need for a method of delivering oral vaccines to animals and presenting large doses of the antigens to mucosal surfaces without having to extract and purify the protein. There is a need to deliver an animal vaccine by directly feeding transgenic plants, plant organs or seeds containing the vaccine antigen to domestic animals. There is a need to provide an immunogenic composition comprising a vaccine antigen in a transgenic plant or seed. The vaccine antigen can be used as oral vaccine in the transgenic plant or seed or extracted and purified for other uses.

### SUMMARY OF THE INVENTION

The present invention provides for transgenic plants which express a foreign protein antigen which when fed to an animal may provide oral immunization against the foreign protein antigen.

According to the invention, an expression cassette for expressing a vaccine antigen in a plant cell is prepared by introducing a DNA sequence which encodes at least one vaccine antigen which is operably linked to transcriptional and translational control regions which function in the plant cell. The vaccine antigen expressed preferably provides protective immunity against mucosal diseases in animals. Preferred expression cassettes of the invention include DNA sequences which encode an antigen from Transmissible Gastroenteritis Virus (TGEV), especially the spiked protein antigen, and porcine rotavirus antigen, especially the VP4 and VP7 antigens.

In one embodiment of the invention, the transcriptional and translational control regions of the expression cassette include a promoter that is inducible. The promoter may include a tissue specific promoter, preferably a seed specific promoter.

The expression cassette of the invention may further comprise a vector. Suitable vectors according to the invention include a binary vector.

In another embodiment, the invention provides a transformed plant cell. Preferably, the transformed plant cell includes an expression cassette which contains a DNA sequence which encodes for a vaccine antigen which is operably linked to transcriptional and translational control regions which are functional in the plant cell. Preferably, the vaccine antigen provides for protection against mucosal disease. According to the invention, the transformed plant cell may be a monocot or dicot plant cell.

In another embodiment of the invention, a transgenic plant is provided which includes an expression cassette which has been stably integrated into the plant genome. Preferably, the expression cassette includes a DNA sequence which encodes for at least one vaccine antigen which is operably linked to transcriptional and translational control regions which function in the plant cell. The transgenic plant may be a monocot or dicot plant. The transcriptional and translational control regions of the plant may include a promoter that provides for a level of gene expression of the vaccine antigen at least about the level which is obtained with the 35S cauliflower mosaic virus promoter. Examples of transgenic plants of the invention include: corn, soybean, sunflower, canola and alfalfa.

The invention also provides for a transgenic plant seed. According to this embodiment, a transgenic plant seed includes an expression cassette which has been stably integrated into the genome of the plant seed. The expression cassette may include a DNA sequence which encodes for at least one vaccine antigen which is operably linked to transcriptional and translational control regions which are functional in the plant seed. Transgenic plant seeds prepared according to the invention include seeds from corn, sunflower, soybeans, alfalfa or canola.

The invention also provides for preparation of an animal feed composition. The animal feed composition of the invention may comprise a transgenic plant or plant seed which includes an expression cassette of the invention.

In a further embodiment of the invention, an immunogenic composition may be prepared. The immunogenic composition may include a transgenic plant or transgenic plant seed which has a vaccine antigen that provides for protection against mucosal disease which is encoded by an expression cassette of the invention. According to the invention, oral administration of an immunogenic composition of the invention may protect an animal against a mucosal disease when the immunogenic composition is administered in an amount effective to provide protection against mucosal disease in an animal. The immunogenic composition of the invention is typically administered by feeding the composition to an animal. The immunogenic composition of the invention may be fed to animals including horses, pigs, cows, sheep, goat, dogs and cats. According to the invention, an effective oral dose of the immunogenic composition is about 0.01-50 mg/kg of body weight.

A further embodiment of the invention provides for an immunogenic composition including a vaccine antigen which provides protection against a mucosal antigen. According to this embodiment of the invention, a transgenic plant is stably transformed with an expression cassette of the invention. The vaccine antigen expressed by the plant is then isolated from the plant and incorporated into a vaccine composition.

### BRIEF DESCRIPTION OF THE DRAWINGS

FIG. 1 is a plasmid map of pPHI5095, an expression vector for the TGEV spike (E2) protein containing the T6 ubiquitin promoter.

FIG. 2 is a plasmid map of pPHI5734, an expression vector for the TGEV spike protein containing the waxy promoter.

FIG. 3 is a plasmid map of pPHI4752, an expression vector for the VP4 or VP7 porcine rotaviruses.

FIG. 4 is a plasmid map of pPHI1680, a binary vector for the VP4 or VP7 proteins of porcine rotaviruses.

FIG. 5 is a plasmid map of pPHI3667, an expression vector for the VP4 or VP7 proteins of porcine rotaviruses containing the napin promoter.

FIG. 6 is a plasmid map of pPHI5765, a binary vector for the VP4 or VP7 proteins of porcine rotaviruses.

FIG. 7 (SEQ ID NO. 1) is a preferred DNA sequence which encodes for the TGEV (E2) spike protein.

### DETAILED DESCRIPTION OF THE INVENTION

According to the present invention, transgenic plants or plant organs, preferably the seeds, are obtained in which a desired animal vaccine antigen is produced. This is achieved via the introduction into the plant of an expression construct comprising a DNA sequence encoding a vaccine antigen and regulatory sequences capable of directing the expression of the antigen in the plant or seeds, preferably the vaccine antigen protects against mucosal diseases in animals. The expression construct provides for the stable transformation of the plants. The transgenic plants or plant organs containing the vaccine antigen may be used as a practical delivery system of the antigen to the animal. Alternatively, the vaccine antigen can be isolated and administered to animals to stimulate active or passive immunity. The vaccine antigen could also be isolated and purified for use in diagnostic assays.

Vaccine antigens, as defined in the context of the present invention, include antigenic or immunogenic components of microorganisms such as viruses, bacteria and parasites intended for the prevention of diseases in animals or that provide protection against diseases in animals. One preferred embodiment of the vaccine is an immunogenic composition comprising transgenic plants or plant organs having an amount of a vaccine antigen or antigens effective to provide protection against diseases, preferably mucosal diseases. Protection against disease includes prevention of infection with the infectious agent, amelioration of the symptoms of the disease, decrease in mortality, induction of secretory IgA, induction of neutralizing antibodies, induction of cell-mediated immunity, or resistance to challenge with virulent organisms. The transgenic plants have an expression construct comprising a DNA sequence encoding the vaccine antigen operably linked to regulatory sequences capable of directing the expression of the vaccine antigen in the plant or plant organs.

The invention also provides for methods of immunizing animals with a vaccine antigen that provides for protection against disease comprising administering an immunogenic composition to an animal wherein the immunogenic composition includes a transgenic plant or seeds having an amount of a vaccine antigen effective to protect animals against disease and is encoded by an expression cassette. Alternatively, the vaccine antigen can form an immunogenic composition after it is isolated from the transgenic plant.

Applicants' methods and compositions are directed toward immunizing and protecting animals, preferably domestic animals, such as cows, sheep, goats, pigs, horses, cats, dogs and llamas. Certain of these animal species can have multiple stomachs and digestive enzymes specific for the decomposition of plant matter, and may otherwise readily inactivate other types of oral vaccines. While not meant to be a limitation of the invention, it is believed that the act of chewing the transgenic plant or feed including transgenic plant material can result in immunization of the animals at the site of the oral mucosa including the tonsils. In addition, the administration of a large dosage of transgenic plant material can allow for the passage of the vaccine antigen containing material to the intestinal tract without being inactivated. Thus, it is believed that transgenic plants having a vaccine antigen can effectively immunize domestic animals via the oral route.

An expression cassette according to the invention comprises a DNA sequence encoding at least one vaccine antigen operably linked to transcriptional and translational control regions functional in a plant cell. The vaccine antigens are preferably selected as antigens that are known to provide protection against mucosal diseases of domestic animals. These vaccine antigens can be derived from viral, bacterial or parasitic sources. This includes cDNA libraries of antigens.

Immunization of animals with these antigens can result in the prevention of infection, amelioration of symptoms, decrease in mortality, induction of a secretory IgA response, and/or induction of neutralizing antibodies. Specific examples of these antigens include the spike (E2) protein of Transmissible Gastroenteritis Virus (TGEV), the VP4 protein of rotaviruses, and the VP7 protein of rotaviruses. Other examples of antigens that may provide protective immunity against mucosal disease include outer membrane proteins (OMB) of Pasteurella haemolytica, Haemophilus somnus and other bacteria, fusion protein of Bovine Respiratory Syncytial Virus (BRSV) or other proteins of viral attachment, Bovine Virus Diarrhea (BVD) antigens, and protective antigens of parasites. Additional antigens important for inducing mucosal immunity or protecting against mucosal disease are known to those of skill in the art.

DNA sequences coding for these antigens can be identified by referring to the published literature or searching a data base of DNA sequences, such as GenBank and the like. Once a DNA sequence coding for a selected vaccine antigen is known, it can be used to design primers and/or probes that are useful in the specific isolation of a DNA or cDNA sequence coding for the vaccine antigen from the pathogen associated with the disease. If a DNA sequence is known, primers and probes can be designed using commercially available software and synthesized by automated synthesis. In general, a DNA sequence coding for a vaccine antigen can be isolated from a library of cDNA or DNA sequences generated from the selected pathogen. The library can be screened for the DNA sequences of interest using a probe complementary to a known DNA sequence encoding a selected antigen, preferably under high stringency conditions. DNA sequences that hybridize to the probe can be subcloned and the polypeptide encoded by the DNA sequence can be confirmed by DNA sequence analysis, in vitro translation, expression and detection of the polypeptide or like assay. Specific examples of DNA sequences coding for a vaccine antigen are sequences coding for spike E2 protein of porcine Transmissible Gastroenteritis Virus Purdue strain, the VP4 protein of the rhesus rotavirus, and the VP7 protein of Nebraska Calf Diarrhea Virus Rotavirus.

Once the DNA sequence coding for at least one vaccine antigen is isolated, it can be operably linked to transcriptional and translational control regions by subcloning into an expression vector. Transcriptional and translational control regions include promoters, enhancers, cis regulatory elements, polyadenylation sequences, transcriptional and translational initiation regions, and transcriptional termination sequences.

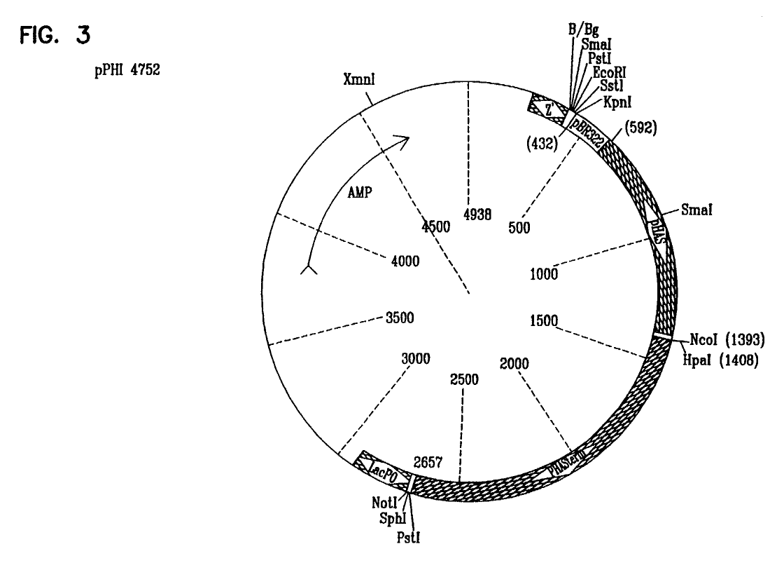

The promoters are preferably those that provide for a sufficient level of expression of a heterologous gene to provide for enough vaccine antigen to immunize an animal orally. The promoters are those that are functional in plants and preferably provide for a level of heterologous gene expression about the same as that provided by the 35S cauliflower mosaic virus (35S CaMV) promoter in the particular plant type. The especially preferred promoters are those that provide for a level of gene expression of about 0.1% to 10% of the total cell protein. Promoters can be inducible, constitutive, or tissue specific. Specific examples of promoters include the 35S CaMV promoter, the nopaline synthase promoter, the chlorophyll A/B binding promoter, the phaseolin promoter, the waxy promoter, the napin promoter, and the ubiquitin promoter. A preferred promoter for the TGEV (E2) spike protein is the phaseolin promoter. See for example, expression cassette pPHI4752 in FIG. 3.

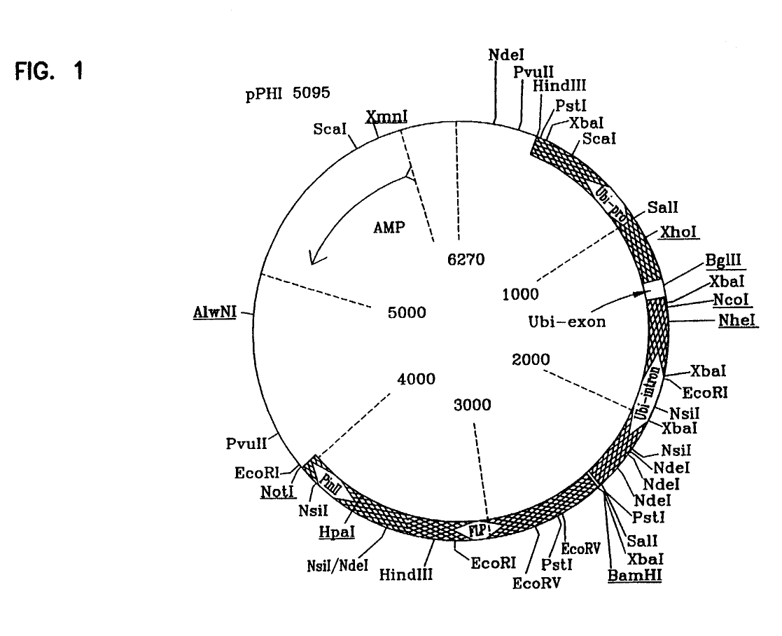

Transcriptional and translational control regions are typically present in expression vectors. Preferably, expression vectors are selected for compatibility and stability in the type of plant cell to be transformed. Some expression vectors including promoters and the 3′ regulatory regions are commercially available such as pCAMVN vector, binary vectors such as pB101 (available from Clone Tech, Palo Alto, Calif. 94303-4230). Preferred vectors include those of FIGS. 1-6 which can be prepared as described in the Examples. Expression vectors can also include those used in amplification and selecting steps such as the baculovirus vector, or phage λ, or other plasmid vectors useful in amplification and cloning of DNA sequences.

Once an expression cassette is formed and subcloned into an appropriate vector system, it can be transformed into suitable host cells. Suitable host cells include bacteria such as E. coli, Agrobacterium tumesfasciens, and plant cells or tissue such as corn suspension cultures, wheat callus suspension cultures, rice protoplast, soy bean tissue, sunflower tissue, alfalfa tissue, and other edible plant cells and tissue. The expression system and vector selected is one that is compatible and stable in the selected host cell. For plant cell transformation, vectors are preferably selected to maximize stable integration of the foreign DNA into the plant cell genome.

Methods of transforming cells depend on the type of host cell selected. For bacterial host cells, methods of transformation include the freeze/thaw method, calcium phosphate precipitation, protoplast transformation, liposome mediated transformation, and electroporation. For plant cell transformation, preferred methods of transformation include agrobacterium mediated transformation, direct transformation of protoplast using electroporation, or direct transfer into protoplast or plant tissue using microparticle bombardment, or combinations of these methods.

Plant cells and tissues to be transformed include those plants useful as animal feed such as alfalfa (Medicago sativa), barley (Hordeum vulgare), beans (Phaseolus spp.), corn (Zea mays), flax (Linum usitatissimum), kapock (Ceiba pentandra), lentil (Lens culinarus), lespedeza (Lespedeza spp.), lupine (Lupinus spp.), sorghum (Sorghum vulgare), mustard and rapeseed (Brassica spp.), oats (Avena sativa), pea (Pisum spp.), peanut (Arachis hupogea), perilla (Perilla spp.), rye (Secala cereale), safflower (Carthamus tinctorius), sesame (Sesamum indicum), soybean (Glycine max), sugar beets (Beta vulgaris saccharifera), sugarcane (Saccharum officinarem), sunflower (Helianthus spp.) and wheat (Triticum aestivum). The choice of plant species is primarily determined by the type of animal being vaccinated. The preferred plant species are corn, soy beans, sunflower, rapeseed, and alfalfa because these represent the major components of most animal feed. Preferably, the protein is expressed in the seed of seed-producing plants such as sunflower. In those plants where the leaves are used as feed, constitutive expression is preferred.

Transformed plant cells are cultured under conditions that select for those cells having the expression cassette, typically by selecting for those cells that exhibit antibiotic resistance. Antibiotic resistance genes are typically used as selectable marker genes. The transformed cells are also grown under conditions that favor regeneration of the cells and/or tissue into plants. Such techniques are known to those of skill in the art and have been described in the Examples. The presence of the desired DNA sequence coding for at least one vaccine antigen in the plant cells or tissues can be determined by hybridization with a probe or by detecting expression by assaying for the presence of the vaccine antigen and other like assays.

Once transgenic plants are obtained, they can be grown under appropriate field conditions until they produce seed. Presence of the DNA sequence coding for the vaccine antigen and expression of the vaccine antigen in the transgenic plant can be determined and quantitated. An expression cassette encoding at least one vaccine antigen is preferably stably integrated into plant cell genome. Stable integration of an expression cassette into a plant cell genome may be established when found in three successive generations. Methods for detection of expression of a protein coded for by the inserted DNA include SDS-page electrophoresis, western blot, ELISA and other methods known in the art. The presence of the DNA sequence coding for the vaccine antigen in the plant genome or chromosomal material can be verified and copy number can be quantitated using hybridization methods known to those of skill in the art. The level of gene expression can be quantitated using quantitative western blots or by measuring the amount of specific mRNA synthesis. Transgenic plants that are expressing the most vaccine antigen as a percentage of the total plant cell protein are preferably selected for further propagation. These plants are preferably expressing the vaccine antigen within the range of 0.1 to 10% of the total plant protein.

Transgenic plants can be crossed with known parental strains and the progeny plants evaluated for the presence of a DNA sequence encoding the vaccine antigen and/or expression of the vaccine antigen. The especially preferred transgenic plants of the invention are those that can transmit the DNA sequence encoding the vaccine antigen to the next generation of plants.

Transgenic seed can be collected from transgenic plants and the level of gene expression of the vaccine antigen in the seed can be determined as described previously. The level of gene expression of the vaccine antigen in the seed is preferably that amount that provides for immunization and/or protection of an animal from mucosal disease. Transgenic seeds that express or contain the vaccine antigen at about 0.1 to 10 percentage of the total seed protein are preferably selected for further propagation.

Transgenic plants, plant organs, and seeds can be combined into animal feed using methods and feed components known to those of skill in the art. The amount of the transgenic plant, plant organ or seed material added to the feed material is that amount that provides sufficient vaccine antigen to an animal to immunize and/or protect the animal against mucosal disease. The amount of vaccine antigen administered in the animal feed will vary depending upon the animal type, the frequency of administration, and the disease.

Transgenic plant, plant organ or seeds containing a vaccine antigen can provide a low cost, easy to administer and distribute vaccine composition. The immunogenic vaccine composition is administered orally to animals, preferably to domestic animals such as the cow, pig, horse, sheep, goat, and poultry. While not meant to limit the invention in any way, it is believed that a vaccine antigen administered in transgenic plant or seeds can immunize animals as they chew at the oral mucosa including the tonsils. In addition, it is known that some of the animal feed can pass through the stomach or stomachs to the intestines undigested or partially digested or that mucosal tissues in the intestine can be exposed to the vaccine antigen. The appropriate range or dose of the transgenic plant material and seed can be determined using standard methodology. The range of dosages of the vaccine antigen for most domestic animals is about 0.01 to 50 mg/kg for oral administration. Once the amount of the vaccine antigen in the transgenic plant or seeds is determined, the amount of transgenic plant or seed material to be administered to the animal can be determined.

The transgenic plants or seeds can be administered by feeding to animals in one or more discrete doses at various time intervals, for example, daily, weekly, monthly, or can be fed continuously. The development of protective immunity can be monitored by detecting the development of specific IgA and/or neutralizing antibodies to the vaccine antigen or a decrease in symptoms or mortality associated with infection with the pathogen.

The vaccine antigen can also be isolated and purified from transgenic plants and/or seeds using standard chromatographic methods. The vaccine antigen can then be used to immunize animals to provide active or passive immunity or can be used in diagnostic assays.

### EXAMPLE 1

### Formation of an Expression Cassette for Expressing TGEV Spike (E2) Protein in Corn

An expression cassette for expression of the TGEV spike (E2) protein in corn can be formed as follows.

The plasmid pPHI5095 as shown in FIG. 1 was prepared. The plasmid contains the T6 ubiquitin promoter and intron with a PinII termination sequence. Between the BamHI and NcoI site is a coding sequence for the heterologous gene, FLP. This coding sequence can be removed by cutting with NcoI and HpaI which will allow other heterologous genes to be inserted by having compatible restriction sites. Alternatively, the gene could be blunt end ligated into the sites or additional cloning sites could be inserted to make this compatible with other genes that provides for constitutive expression of a heterologous gene under control of the ubiquitin promoter. This plasmid has been used successfully to provide for expression of FLP, β-glucuronidase and wheat germ agglutinum (WGA), genes in maize cells.

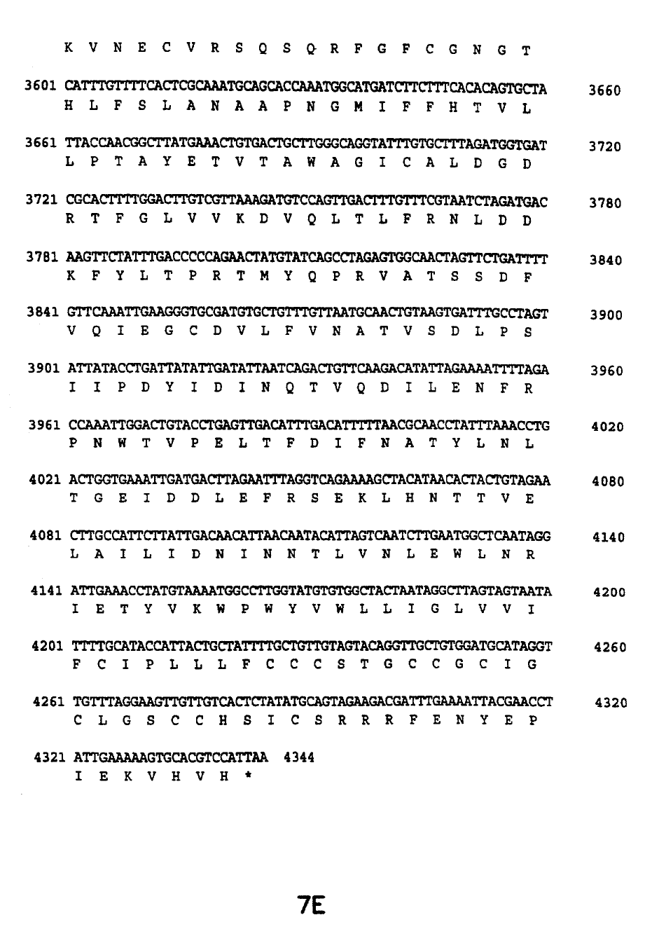

A DNA sequence coding for the TGEV spike (E2) protein is known, (Vaughn et al., J. Virol., 69:3176 (1995), Rasschaert et al., J. Gen. Virol., 68:1883 (1987) or can be obtained using standard techniques as described in Maniatis et al., A Guide to Molecular Cloning, Cold Spring Harbor, N.Y. (1989). A preferred DNA sequence coding for the TGEV spike (E2) protein is shown in FIGS. 7A-E.

Briefly, cDNA can be prepared from genomic RNA using reverse transcriptase and oligo dT primers or a specific primer designed from the known DNA sequence. Double stranded cDNA can be dC-tailed using terminal transferase and annealed to a dG-tailed restriction endonuclease cleaved vector. The vectors can be introduced into a bacterial host cell, and transformants carrying viral inserts can be identified using probes designed for the known DNA sequence or by using antibodies specific for the TGEV (E2) spike protein.

Once the DNA sequence coding for the TGEV (E2) spike protein is isolated it can be subcloned into vectors such as the modified pPHI5095 at BamHI and HpaI sites so that the expression of this DNA sequence is under control of the ubiquitin promoter. Plasmids including the DNA sequence coding the TGEV spike protein can be selected by examining the restriction digest patterns from plasmids that were isolates from cells growing on ampicillin.

### EXAMPLE 2

### Preparation of Transgenic Corn Having an Expression Cassette Coding for the TGEV (E2) Spike Protein

Once formed a vector carrying a DNA sequence coding for the TGEV (E2) spike protein under control of a promoter functional in the plant can be used to form transgenic corn plants. A method for formation of transgenic corn plants has been described in European Patent Application No. 0 442 174A1 which is hereby incorporated by reference. A brief description of that methodology follows.

A vector carrying a DNA sequence coding for a TGEV (E2) spike protein formed as described in Example 1 can be introduced into corn tissue or suspension cells by microparticle bombardment. In addition, a construct containing a 35S expression cassette can be cotransformed with the TGEV spike protein to all for easy selection of transformed plants. The 35S cassette is disclosed in Gordon-Kamm et al., The Plant Cell, 2:603-18 (1990). 35S contains the BAR gene which has been shown to give resistance to cells for glufosinate selective agents.

Preferably, germ cells are used including those derived from a meristem of immature embryos. Suspension cell lines are also available to generate embryogenic suspension cultures. For example, embryogenic suspension cultures can be derived from type II embryogenic culture according to the method of Green et al., Molecular Genetics of Plants and Animals, editors Downey et al., Academic Press, New York 20, 147 (1983). The callus can be initiated from maize inbreds designated R21 and B73×G35. Both R21 and G35 are proprietary inbred lines developed by Pioneer Hybred International Inc. Des Moines, Iowa. Suspension cultures of the cultivar “Black Mexican Sweet” (“BMS”) can be obtained from Stanford University. The cultures can be maintained in Murashige and Skoog (“MS”) medium as described in Murashige et al., Physio. Plant, 15:453-497 (1962) supplemented with 2,4-dichlorophenoxyacidic acid (2,4-D) at 2 mg/L and sucrose at 30 g/L. The suspension cultures are passed through a 710 micron sieve 7 days prior to the experiment and filtrate can be maintained in MS medium. In preparation for microparticle bombardment, cells are harvested from the suspension culture by vacuum filtration on a Buchner funnel (Whatman No. 614). Alternatively, callus cells can be passed through a sieve and used for bombardment.

Prior to the microparticle bombardment, a 100 ml (fresh weight) of cells are placed in a 3.3 cm petri plate. The cells are dispersed in 0.5 mL fresh culture medium to form a thin layer of cells. The uncovered petri plate is placed in the sample chamber of a particle gun device manufactured by Biolistics Inc., Geneva, N.Y. A vacuum pump is used to reduce the pressure in the chamber to 0.1 atmosphere to reduce deceleration of the microparticles by air friction. The cells are bombarded with tungsten particles having an average diameter of about 1.2 microns, obtained from GTE Sulvania Precision Materials Group, Towanda, Pa. The microparticles have a DNA loading consisting of equal mixtures of the selectable and nonselectable plasmids. The DNA is applied by adding 5 μl of 0.1 g % solution of DNA in TE buffer at pH 7.7 to 25 μl of a suspension of 50 mg of tungsten particles per ml distilled water in a 1.5 ml Eppendorf tube. Particles become agglomerated and settle.

Cultures of transformed plant cells containing the foreign gene are cultivated for 4-8 weeks in 560R medium (N6-based medium with 3 mg/l of bialophos). After this time, only cells that received the BAR gene are able to proliferate. These events are rescued and identified as transformants. The putative transformants are then tested for the presence of integration of TGEV DNA by PCR. Transient expression of the DNA sequence coding for the TGEV (E2) spike protein at 24-72 hours after bombardment can be detected using western blots, ELISA and antibodies to the TGEV spike protein.

Embryo formation can then be induced from the embryogenic cultures to the stage of maturing and germination into plants. A two culture medium sequence is used to germinate somatic embryos observed on callus maintenance medium. Callus is transferred first to a culture medium (maturation medium) which instead of a 0.75 mg/L, 2,4-D has 5.0 mg/L indoleacetic acid (IAA). The callus culture remains on this medium for 10 to 14 days while callus proliferation continues at a slower rate. At this culture stage, it is important that the amount of callus started on the culture medium not be to large or fewer plants be recovered per unit mass of material. Especially preferred is an amount of 50 mg of callus per plate.

Toward the end of this culture phase, observation under a dissecting microscope often indicates somatic embryos have begun germinating although they are white in color because this culture phase is done in darkness.

Following this first culture phase, callus is transferred from “maturation” medium to a second culture medium which further promotes germination of the somatic embryos into a plantlet. This culture medium has a reduced level of IAA versus the first culture medium, preferably a concentration of about 1 mg/L. At this point, the cultures are placed into the light. Germinating somatic embryos are characterized by a green shoot which elongates often with a connecting root access. Somatic embryos germinate in about 10 days and are then transferred to medium in a culture tube (150×25 mm) for an additional 10-14 days. At this time, the plants are about 7⁻¹⁰ cm tall, and are of sufficient size and vigor to be hardened off to greenhouse conditions.

To harden off regenerated plants, plants are removed from the sterile containers and solidified agar medium is rinsed off the roots. The plantlets are placed in a commercial potting mix in a growth chamber with a misting device which maintains the relative humidity near 100% without excessively wetting the plant roots. Approximately 3 or 4 weeks are required in the misting chamber before the plants are robust enough for transplantation into pots or into field conditions. At this point, many plantlets especially those regenerated from short term callus cultures will grow at a rate into a size similar to seed derived plants. Ten to fourteen days after pollination, the plants are checked for seed set. If there is seed, the plants are then placed in a holding area in the green house to mature and dry down. Harvesting is typically performed 6 to 8 weeks after pollination.

This methodology has been used successfully to regenerate corn plants expressing the chloramphenicol acetotransferase gene under control of the 35S cauliflower mosaic virus (35S CaMV) promoter as well as many other sized genes. Direct introduction of foreign DNA into suspension culture or tissues of monocot plants has been used successfully for regenerating transgenic monocot plants such as corn, wheat, rice and the like.

### EXAMPLE 3

### Formation of Transgenic Corn Seeds Carrying an Expression Cassette Coding for the TGEV (E2) Spike Protein

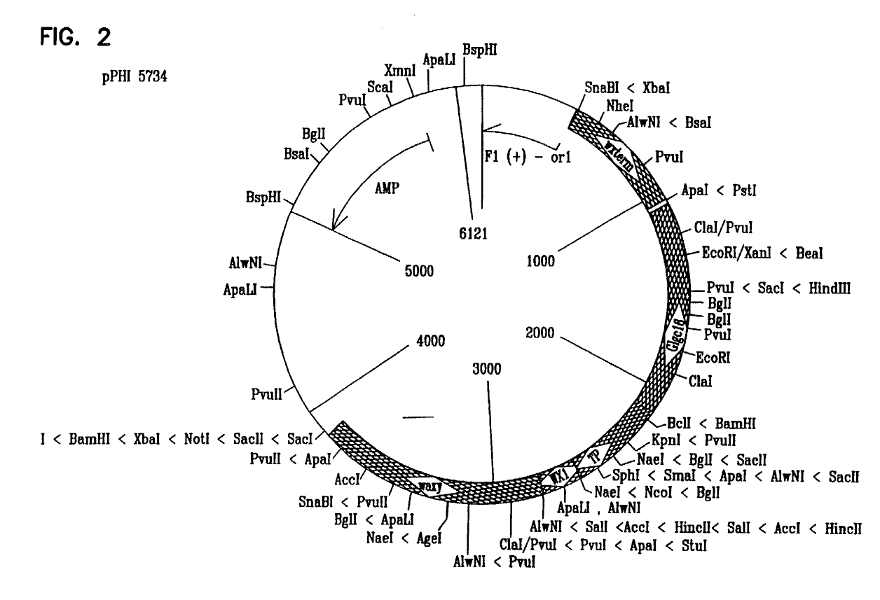

The DNA sequence coding for the spike protein of the TGE virus can be inserted into an expression cassette under control of the waxy promoter for seed specific expression. A cassette is present in a vector such as a plasmid pPHI5734 as shown in FIG. 2.

Plasmid pPHI5734 has the waxy regulatory sequences and a heterologous gene coding sequence and can be inserted between the NcoI and PstI sides. Alternatively, the heterologous gene can be blunt end ligated or additional cloning cites can be added to make them compatible with the coding sequence of the heterologous gene.

A DNA sequence coding of the TGEV (E2) spike protein can be obtained as described in Example 1. This DNA sequence can be inserted into the multiple cloning site at NcoI and PstI in plasmid pPHI5734 using standard methods. A plasmid including a DNA sequence coding for the TGEV (E2) spike protein under control of a seed specific promoter can be selected and isolated by examining the restriction patterns of the recombinant plasmid and sequencing.

Corn cells are transformed by microparticle bombardment as described in Example 2. Transformed cells containing a DNA sequence coding for the TGEV (E2) spike protein can be identified and selected by PCR. Transgenic corn plants and seeds can be regenerated as described in Example 2. Expression of TGEV (E2) spike protein in seeds can be confirmed and quantitated by ELISA or western blot analysis. Stability of the expression of the TGEV spike (E2) protein can be evaluated by these same methods over successive generations.

### EXAMPLE 4

### Formation of an Expression Cassette Encoding VP4 and VP7 Proteins of Porcine Rotavirus

An expression cassette can be formed for expression of the VP4 and/or VP7 proteins of porcine rotavirus under control of the promoter for the seed storage protein phaseolin. The expression cassette can be formed with a DNA sequence encoding VP4 and a DNA sequence encoding VP7 under control of the single promoter to form a dicistronic construct or each DNA sequence can be placed under control of its own promoter but the same promoter. The expression cassette is present in a vector such as the pPHI4752 shown in FIG. 3.

Plasmid pPHI4752 was prepared by linking the phaseolin upstream regulator region adjacent to the downstream region of the phaseolin gene, but not including the coding sequence of the gene itself.

Plasmid pPHI4752 has a NcoI and HpaI site that can be used to insert heterologous genes downstream from the phaseolin promoter. The phaseolin promoter has been used successfully to express the Brazil nut protein, in soybean, canola and tobacco.

A DNA sequence coding for the VP4 protein of porcine rotavirus can be obtained using standard methods as described in Maniatis et al., cited supra. A DNA sequence encoding VP4 can also be obtained as described by Mackow et al., Gen. Virol., 63:1661 (1989). Briefly, cDNA synthesis of genomic RNA can be conducted using reverse transcriptase and specific primers such as those representing the 5′ end of each strand of gene 4 double stranded RNA or primers can be designed from a known DNA sequence for VP4. Double stranded cDNA synthesis can be performed and adaptors can be ligated onto the ends of the cDNA sequence to provide for ease of cloning into a vector. The cDNA sequences can then be introduced into a vector such as phage λ and amplified in bacterial host cells. Transformants containing viral inserts can be screened by hybridization to a probe designed based on a known DNA sequence for VP-4. Once the DNA sequence encoding VP-4 is isolated, it can be introduced into an expression vector such as the baculovirus vector.

Once obtained in a vector such as the baculovirus vector, the DNA sequence can be subcloned into pPHI4752 at a cloning site NcoI and HpaI so that its expression is controlled by the phaseolin promoter. Plasmid pPHI4752, including a DNA sequence encoding VP4, can be selected, amplified and isolated by examining the restriction digestion patterns of plasmids from cells growing in kanamycin.

The DNA sequence coding for VP7 can be obtained by the method as described in Grass et al., Virology, 141:292 (1985). Briefly, mRNA from virus propagated into a host cell is isolated, poly-A tailed and reverse transcribed with oligo dT priming. Single stranded cDNAs are tailed at 3′ ends with oligo d(c) and primered with oligo d(G) and transcribed with reverse transcriptase. Double stranded cDNAs are inserted at a restriction endonuclease site of a vector. The vectors are then transformed into a bacterial host cell. Transformants having viral inserts encoding VP-7 can be identified by hybridization to probes designed from the known sequence of VP-7. Once isolated and identified, cDNA sequence encoding VP-7 can be subcloned from a plasmid such as pBR322 to a binary vector.

Once obtained in a vector such as the pBR322, the DNA sequence coding for VP7 can be subcloned in a plasmid pPHI4752 at cloning site NcoI and HpaI so that its expression is controlled by the phaseolin promoter. Alternatively, it can be subcloned immediately downstream from the DNA sequence coding for VP4 to form a dicistronic construct under control of a single phaseolin promoter. Plasmid pPHI4752, including a DNA sequence encoding VP4 can be selected, amplified and isolated as above.

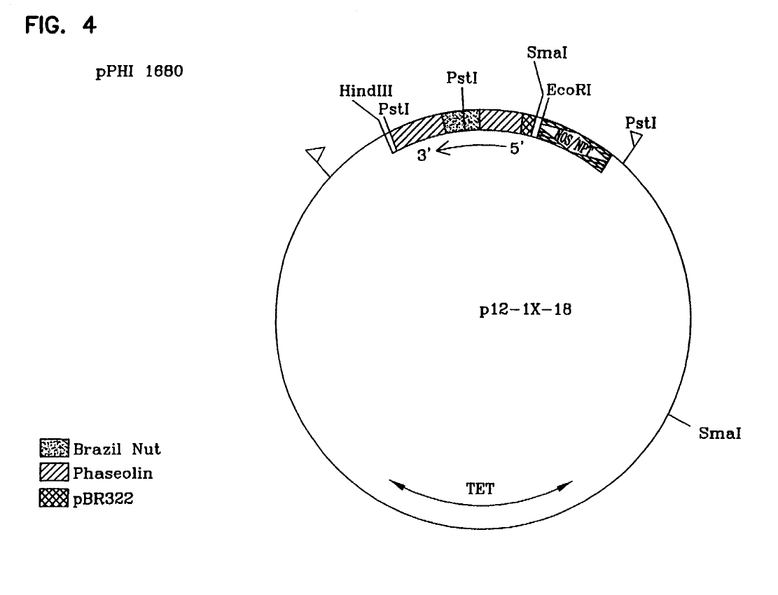

The expression cassette can then be subcloned into a binary vector such as pPHI1680 at the EcoRI and HinD III. See FIG. 4. This binary vector is available at Pioneer Hybrid International, Inc., Johnston, Iowa 50131. The binary vector carrying the expression cassette coding for VP4 and or VP7 is introduced into Agrobacterium tumesfasciens tumafocious strain LBA4404 (available from Clone Tech, Palo Alto, Calif. 94303-4230) or other disarmed A. tumesfaciens strains by the freeze thaw method.

### EXAMPLE 5

### The Agrobacterium Strains having a Binary Vector Including a DNA Sequence Encoding VP4 or VP7 of Porcine Rota Virus can be used to Form Transgenic Soybean Plants

A method for forming transgenic soybean plants is that described in U.S. patent application Ser. No. 07/920,409 which is hereby incorporated by reference. Soybean (glycine max) seed, of Pioneer variety 9341 is surface sterilized by exposure to chlorine gas evolved in a glass bell jar. Gas is produced by adding 3.5 ml hydrochloric acid (34 to 37% w/w) to 100 ml sodium hypochlorite (5.25% w/w). Exposure is for 16 to 20 hours in a container approximately 1 cubic ft in volume. Surface sterilize seed is stored in petri dishes at room temperature. Seed is germinated by plating 1/10 strength agar solidified medium according to Gambourg (B5 basal medium with minimal organics, Sigma Chemical Catalog No. G5893, 0.32 gm/L sucrose; 0.2% weight/volume and 2-(N-morpholino)ethanesulfonic acid (MES), 3.0 mM) without plant growth regulators and culturing at 28° with a 16-hour day length and cool white florescent illumination of approximately 20 μEM² S¹. After 3 or 4 days, seed is prepared for co-cultivation. The seed coat is removed and the elongating radical is removed 3 to 4 mm below the cotyledons.

Overnight cultures of Agrobacterium tumesfasciens strain LBA4404 harboring the modified binary plasmid pPHI1680 (FIG. 4) are grown to log phase in minimal A medium containing tetracycline, 1 μg/ml, are pooled and an optical density measurement at 550 nanometers is taken. Sufficient volume of the culture is placed in 15 m/conical centrifuge tubes such that upon sedimentation between 1 and 2×10¹⁰ cells were collected in each tube where DD=55, 1=1.4×10⁹ cells/ml. Sedimentation is by centrifugation at 6,000×g for 10 min. After centrifugation, the supernatant is decanted and the tubes are held at room temperature until inoculum is needed but not longer than 1 hour.

Inoculations are conducted in batches such that each plate of seed is treated with a newly resuspended pellet of Agrobacterium. One at a time the pellets are resuspended in 20 ml inoculation medium. Inoculation medium consisted of B5 salts (G5893), 3.2 g/L; sucrose, 2.0% w/v; 6-benzylaminopurine (BAP), 45 λm; indolebutyric acid (IBA), 0.5 μM; acetosyringone (AS), 100 μM; and was buffered to pH 5.5 with MES 10 mM. Resuspension is by vortexing. The inoculum is then poured into a petri dish containing a prepared seed and the cotyledonary nodes are masserated with surgical blade. This is accomplished by dividing seed in half by longitudinal section through the shoot apex preserving the 2 whole cotyledons. The two halves of shoot apex are then broken off their respective cotyledons by prying them away with a surgical blade. The cotyledonary node is then macerated with surgical blade by repeated scoring along the axis of symmetry. Care was taken not to cut entirely through the explant to the abaxial side. Explants are prepared in roughly about 5 min and then incubated for 30 minutes at room temperature without agitation. After 30 minutes, the explants are transferred into plates of the same medium solidified with Gelrite (Merck & Company Inc.), 0.2% w/v. Explants are imbedded with adaxial side up and leveled with the surface of the medium and cultured at 22° C. for 3 days under cool white fluorescent light, approximately 20 EM²S¹.

After 3 days, the explants are moved to liquid counterselection medium. Counterselection medium consisted of B5 salts (G5893), 3.2 g/l; sucrose, 2% w/v; BAP, 5 μM; IBA, 0.5 μM; vancomycin, 200 μg/ml; cefotaxime, 500 μg/ml and was buffered to pH 5.7 with MES, 3 mM. Explants are washed in each petri dish with constant slow gyratory agitation at room temperature for 4 days. Counterselection medium is replaced 4 times.

The explants are then picked to agarose/solidified selection medium. The selection medium consisted of B5 salts (G5893), 3.2 g/l; sucrose, 2% w/v; BAP, 5.0 μM; IBA, 0.5 μM; kanamycin sulfate, 50 μg/ml; vancomycin, 100 μg/ml; cefotaxime, 30 μg/ml; timentin, 30 μg/ml and is buffered to pH 5.7 with MES, 3mM. Selection medium was solidified with Seakem Argarose, 0.3 w/v. The explants are imbedded in the medium, adaxial side down and cultured at 28° with a 16 hour day length in cool white florescent illumination of 60 to 80μ EM²S¹.

After 2 weeks explants are again washed with liquid medium on the gyratory shaker. The wash is conducted overnight in counterselection medium containing kanamycin sulfate, 50 μg/ml. The following day, explants are picked to agarose/solidified selection medium. They are imbedded in the medium at adaxial side down and cultured for another 2 week period.

After 1 month on selected medium, transformed tissue is visible as green sectors of regenerating tissue against a background of bleachless healthy tissue. Explants without green sectors are discarded, explants with green sectors are transferred to elongation medium. Elongation medium consists of BS salts (G5893), 3.2 g/l; sucrose, 2% w/v; IBA, 3.3 μM; gibberellic acid, 1.7 μM; vancomycin, 100 μg/ml; cefotaxime, 30 μg/ml; and tomentin, 30 μg/ml, buffered to pH 5.7 with MES, 3 mM. Elongation medium is solidified with Gelrite, 0.2% w/v. The green sectors are imbedded at adaxial side up and cultured as before. Culture is continued on this medium with transfers to fresh plates every two weeks. When shoots are 0.5 cm in length they are excised at the base and placed in rooting medium in 13×100 ml test tubes. Rooting medium consisted of B5 salts (G5893), 3.2 g/l; sucrose, 15 g/l; nicotinic acid, 20 μm; pyroglutamic acid (PGA), 900 mg/L and IBA 10 μM. The rooting medium is buffered to pH 5.7 with MES 3 mM and solidified with Gelrite 0.2% w/v. After 10 days, the shoots are transferred to the same medium without IBA or PGA. Shoots are rooted and held in these tubes under the same environmental conditions as before.

When a root system was well established the plantlet is transferred to sterile soil mixed in plantcons. Temperature, photoperiod and light intensity remain the same as before.

The expression of VP4 and/or VP7 in transgenic soybean plants can be confirmed by PCR and quantitated using ELISA or western blot analysis. Stability of expression can be evaluated by these same methods over successive generations.

### EXAMPLE 6

### Formation of an Expression Cassette and Transgenic Sunflower Plant and Seeds Including the VP4 and/or VP7 Proteins of Porcine Rota Virus

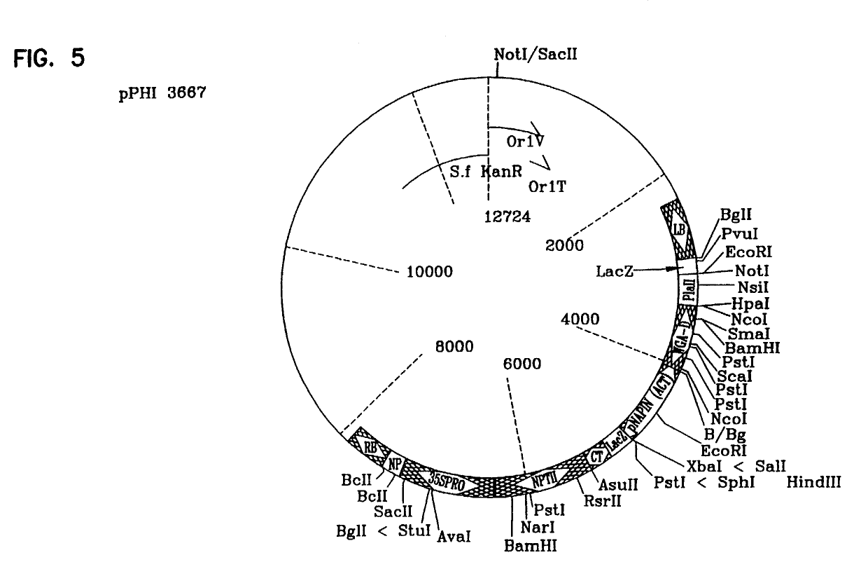

An expression cassette encoding VP4 and/or VP7 can be used to generate transgenic sunflower seeds and plants. The DNA sequence coding for VP4 and/or VP7 can be inserted into an expression cassette under control of the napin promoter for seeds specific expression. The expression cassette is present in a vector such as a plasmid pPHI3667 as shown in FIG. 5.

Plasmid pPHI3667 was prepared by aligning the napin promoter region upstream to the coding region of the heterologous gene and the PinII termination sequence downstream.

The characteristics of plasmid pPHI3667 include a plant transcription unit for the gene NPTII which can be used in selecting transformed cells. The plasmid pPHI3667 has a NcoI and HpaI cloning site that provides for seed specific expression under control of the napin promoter. This promoter has been used successfully to express WGA and, β-glucuronidase genes in canola seeds.

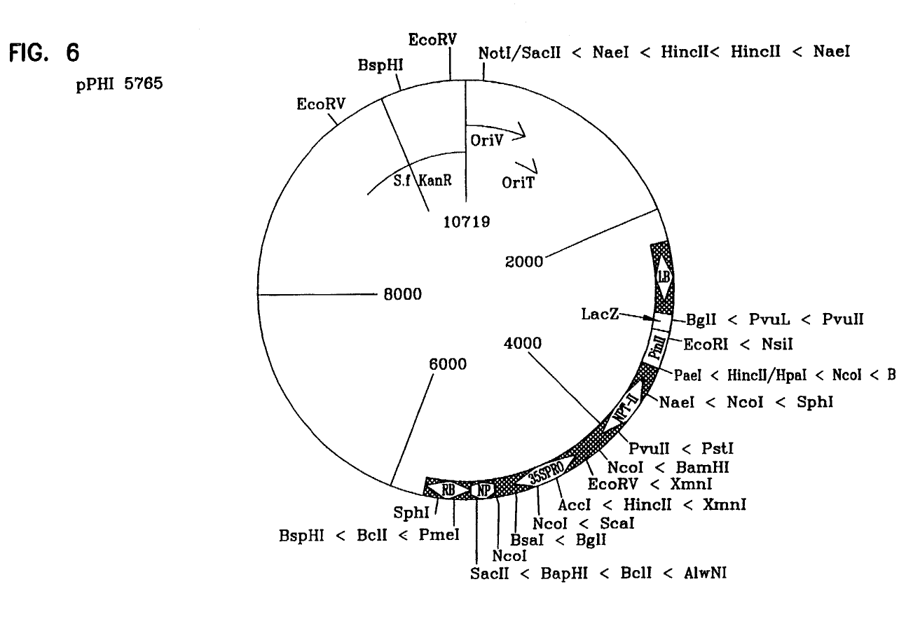

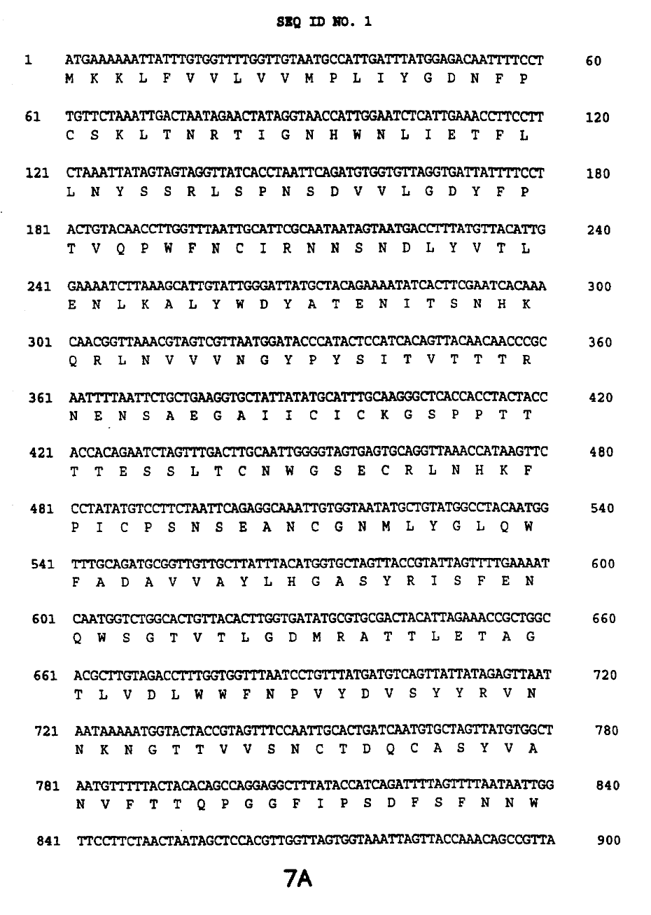

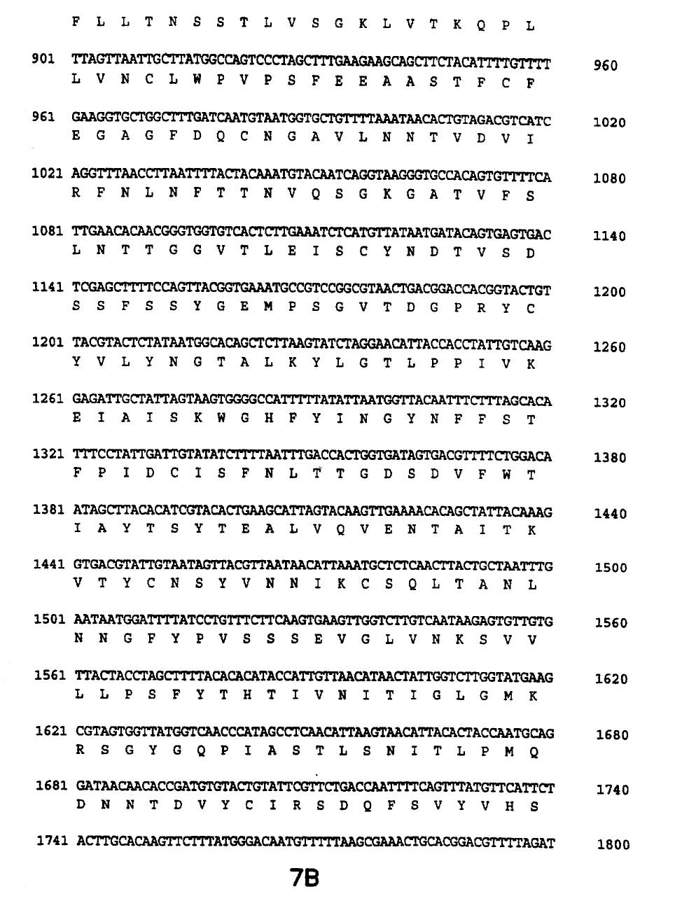

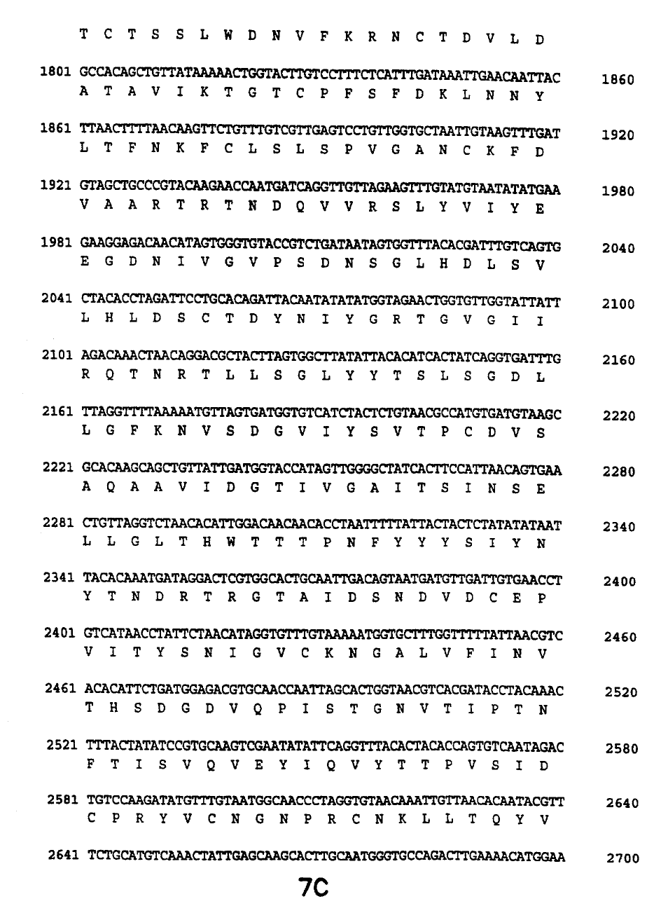

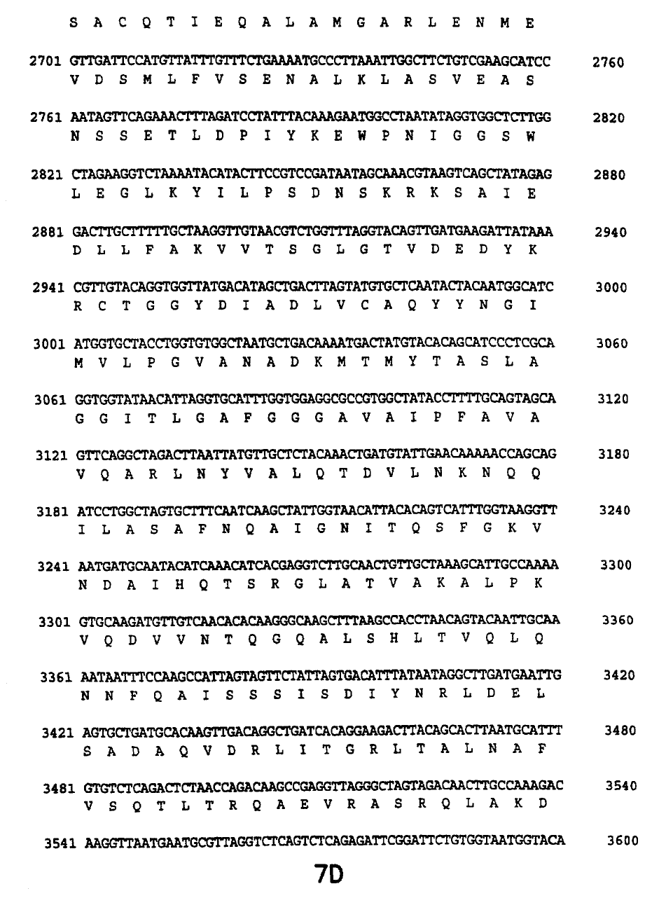

A DNA sequence encoding VP4 and/or VP7 can be obtained as described in Example 5. The DNA sequence can be subcloned into the NcoI or HpaI site in pPHI3667. Plasmids having a DNA sequence encoding VP4 and/or VP7 can be selected, amplified and isolated by using phage cDNA libraries as described in Maniatis et al., A Guide to Molecular Cloning, Cold Spring Harbor, N.Y. (1989). This expression cassette is then subcloned into a binary vector such as pPHI5765 using the EcoRI site in Agrobacterium tumesfasciens strain LBA4404. See FIG. 6.

Sunflower plants can be transformed with Agrobacterium strain LBA4404 by the method of microparticle bombardment as described by Bidney et al., Plant Mol. Bio., 18:301 (1992). Briefly, seeds of Pioneer Sunflower Line SMF-3 are dehulled and surface sterilized. The seeds are imbibed in the dark at 26° C. for 18 hours on filter paper moistened with water. The cotyledons and root radical are removed and meristem explants cultured on 374BGA medium (MS salts, Shephard vitamins, 40 ml/L adenine sulfate, 3% sucrose, 0.8% phytagar pH 5.6 plus 0.5 mg/L of BAP, 0.25 ml/L, IAA and 0.1 mg/L GA). Twenty-four hours later, the primary leaves are removed to expose the apical meristem and the explants are placed with the apical dome facing upward in a 2 cm circle in the circle of a 60 mM by 20 mM petri plate containing water agar. The explants are bombarded twice with tungsten particles suspended in TE buffer as described above or with particles associated with plasmid pPHI3667. Some of the TE/particle bombardment explants are further treated with Agrobacterium tumesfasciens strain carrying pPHI3667 by placing a droplet of bacteria suspended in the inoculation medium, OD600 2.00, directly onto the meristem. The meristem explants are co-cultured on 374BGA medium in the light at 26° C. for an additional 72 hours.

Agrobacterium treated meristems are transferred following the 72 hour co-culture period to medium 374 (374BGA with 1% sucrose plus 50 mg/l kanamycin sulfate and no BAP, IAA or GA₃) and supplemented with 250 μg/ml cefotaxime. The plantlets are allowed to develop for an additional 2 weeks under 16 hour day and 26° C. incubation conditions. Green or unbleached plantlets are transferred to medium 374 and grown until they develop seed. The presence of VP4 and VP7 in sunflower plants and seeds can be confirmed and quantitated as described in Example 5.

### EXAMPLE 7

### Immunization of Pigs Against TGEV Virus

Transmissible Gastroenteritis Virus (TGEV) causes an acute and fatal enteric disease in newborn piglets. In adult pigs, the infection with the virus is characterized by anorexia, dehydration, severe diarrhea followed by death. Pigs at 5-7 days old will be fed canola or corn oil which includes the TGEV spike E2 protein in order to immunize and protect the pigs from enteric disease and symptoms caused by the TGE virus.

The transgenic canola or corn plant carrying an expression cassette comprising a DNA sequence coding for TGEV (E2) spike protein can be formed as described in Example 2. The levels of expression of the TGEV (E2) spike protein in the seed can be assessed using quantitative western blots with monoclonal antibodies to the TGEV (E2) spike protein. Once the level of expression of the TGEV (E2) spike protein in the seed is quantitated, the amount of transgenic plant material to be administered to the animal to achieve doses in the range of 0.01 to 50 mg/kg can be determined.

A standard dose response immunization schedule can be employed to determine the optimal dosages for oral immunization to induce protection against TGE virus. Groups of pigs 5-7 days old will be fed different doses such as 0.1, 1.0, 5.0, and 25.0 mg/kg of the TGEV (E2) spike protein daily for 5 days. The development of protective immunity in the pigs can be evaluated by examining the pigs for the development of neutralizing antibodies and/or IgA antibodies to TGEV (E2) spike protein. Immunized pigs can also be challenged with the TGE virus and the level of infection and symptoms such as diarrhea or death can be monitored. It is expected that as the dosage of the TGEV (E2) spike protein in the seed is increased, there will be an increase in the observed protective effect, the formation of neutralizing antibodies, and/or the formation of IgA antibodies to the TGEV (E2) spike protein.

What is claimed is:

1. An expression cassette for expressing a vaccine antigen in a plant cell comprising a DNA sequence encoding at least one vaccine antigen operably linked to transcriptional and translation control regions functional in the plant cell, wherein the vaccine antigen provides protection against mucosal diseases.

2. The expression cassette according to claim 1, wherein the DNA sequence encodes an antigen from Transmissible Gastroenteritis Virus (TGEV).

3. The expression cassette according to claim 2, wherein the antigen is the spike protein.

4. The expression cassette according to claim 1, wherein the DNA sequence encodes an antigen from porcine rotavirus.

5. The expression cassette according to claim 4, wherein the antigen is VP4.

6. The expression cassette according to claim 4, wherein the antigen is VP7.

7. The expression cassette according to claim 1, wherein the transcriptional and translation control regions comprise a promoter that is inducible.

8. The expression cassette according to claim 1, wherein the transcriptional and translation control regions comprise a tissue specific promoter.

9. The expression cassette according to claim 1, wherein the transcriptional and translational control regions comprise a seed specific promoter.

10. The expression cassette according to claim 1 further comprising a vector.

11. The vector according to claim 10, wherein the vector is a binary vector.

12. A transformed plant cell comprising an expression cassette comprising a DNA sequence encoding for a vaccine antigen operably linked to transcriptional and translational control regions functional in the plant cell, wherein the vaccine antigen provides for protection against mucosal disease.

13. The transformed plant cell according to claim 12, wherein the cell is a monocot.

14. The transformed plant cell according to claim 12, wherein the plant cell is a dicot.

15. The transformed plant cell according to claim 12, wherein the DNA sequence encodes an antigen from Transmissible Gastroenteritis Virus (TGEV).

16. A transgenic plant comprising an expression cassette stably integrated into the plant genome wherein the expression cassette comprises a DNA sequence encoding at least one vaccine antigen operably linked to transcriptional and translational control regions functional in the plant cell, wherein the vaccine antigen provides protection against mucosal disease.

17. The transgenic plant according to claim 16, wherein the plant is a monocot.

18. The transgenic plant according to claim 16, wherein the transcriptional and translational control regions comprises a promoter that provides for a level of gene expression of the vaccine antigen at least about the level obtained with the 35S cauliflower mosaic virus promoter.

19. The transgenic plant according to claim 17, wherein the plant is corn, soybeans, sunflower, canola or alfalfa.

20. A transgenic plant seed comprising:
an expression cassette stably integrated into the genome of the plant seed and comprising a DNA sequence encoding at least one vaccine antigen operably linked to transcriptional and translational control regions functional in the plant seed, wherein the vaccine antigen provides for protection against mucosal disease.

21. The transgenic seed according to claim 20, wherein the plant seed is selected from the group of corn, sunflower, soybeans or canola.

22. An animal feed composition comprising a transgenic plant or seed, wherein the transgenic plant or seed comprise an expression cassette of claim 1.

23. An immunogenic composition comprising a transgenic plant or seed having a vaccine antigen that provides for protection against mucosal disease and which is encoded by an expression cassette according to claim 1.

24. A composition according to claim 23 further comprising an adjuvant.

25. A method for protecting an animal against mucosal disease comprising administering orally an immunogenic composition according to claim 23 in an amount effective to provide protection against mucosal disease to an animal.

26. The method according to claim 25, wherein the immunogenic composition is administered by feeding the immunogenic composition to an animal.

27. The method according to claim 25, wherein the animal is a pig, cow, sheep, goat, dog or cat.

28. The method according to claim 25, wherein an effective amount is a dose range of 0.01 to 50 mg per kg of bodyweight.

29. An immunogenic composition comprising a vaccine antigen, wherein the vaccine antigen provides for protection against mucosal disease in an animal and which is produced by the process comprising:
a) forming a transgenic plant expressing the vaccine antigen by stably transforming the plant with an expression cassette comprising a DNA sequence encoding the vaccine antigen operably linked to transcriptional and translational control regions functional in the plant; and
b) isolating the vaccine antigen from the plant.


SEQUENCE LISTING (2 sequences)

SEQ ID NO: 1 - DNA, 4344 bp, Transmissible Gatroenteritis Virus Spike protein
  CDS (1)..(4344)
atgaaaaaattatttgtggttttggttgtaatgccattgatttatggagacaattttccttgttctaaattgactaatagaactataggtaaccattggaatctcattgaaaccttccttctaaattatagtagtaggttatcacctaattcagatgtggtgttaggtgattattttcctactgtacaaccttggtttaattgcattcgcaataatagtaatgacctttatgttacattggaaaatcttaaagcattgtattgggattatgctacagaaaatatcacttcgaatcacaaacaacggttaaacgtagtcgttaatggatacccatactccatcacagttacaacaacccgcaattttaattctgctgaaggtgctattatatgcatttgcaagggctcaccacctactaccaccacagaatctagtttgacttgcaattggggtagtgagtgcaggttaaaccataagttccctatatgtccttctaattcagaggcaaattgtggtaatatgctgtatggcctacaatggtttgcagatgcggttgttgcttatttacatggtgctagttaccgtattagttttgaaaatcaatggtctggcactgttacacttggtgatatgcgtgcgactacattagaaaccgctggcacgcttgtagacctttggtggtttaatcctgtttatgatgtcagttattatagagttaataataaaaatggtactaccgtagtttccaattgcactgatcaatgtgctagttatgtggctaatgtttttactacacagccaggaggctttataccatcagattttagttttaataattggttccttctaactaatagctccacgttggttagtggtaaattagttaccaaacagccgttattagttaattgcttatggccagtccctagctttgaagaagcagcttctacattttgttttgaaggtgctggctttgatcaatgtaatggtgctgttttaaataacactgtagacgtcatcaggtttaaccttaattttactacaaatgtacaatcaggtaagggtgccacagtgttttcattgaacacaacgggtggtgtcactcttgaaatctcatgttataatgatacagtgagtgactcgagcttttccagttacggtgaaatgccgtccggcgtaactgacggaccacggtactgttacgtactctataatggcacagctcttaagtatctaggaacattaccacctattgtcaaggagattgctattagtaagtggggccatttttatattaatggttacaatttctttagcacatttcctattgattgtatatcttttaatttgaccactggtgatagtgacgttttctggacaatagcttacacatcgtacactgaagcattagtacaagttgaaaacacagctattacaaaggtgacgtattgtaatagttacgttaataacattaaatgctctcaacttactgctaatttgaataatggattttatcctgtttcttcaagtgaagttggtcttgtcaataagagtgttgtgttactacctagcttttacacacataccattgttaacataactattggtcttggtatgaagcgtagtggttatggtcaacccatagcctcaacattaagtaacattacactaccaatgcaggataacaacaccgatgtgtactgtattcgttctgaccaattttcagtttatgttcattctacttgcacaagttctttatgggacaatgtttttaagcgaaactgcacggacgttttagatgccacagctgttataaaaactggtacttgtcctttctcatttgataaattgaacaattacttaacttttaacaagttctgtttgtcgttgagtcctgttggtgctaattgtaagtttgatgtagctgcccgtacaagaaccaatgatcaggttgttagaagtttgtatgtaatatatgaagaaggagacaacatagtgggtgtaccgtctgataatagtggtttacacgatttgtcagtgctacacctagattcctgcacagattacaatatatatggtagaactggtgttggtattattagacaaactaacaggacgctacttagtggcttatattacacatcactatcaggtgatttgttaggttttaaaaatgttagtgatggtgtcatctactctgtaacgccatgtgatgtaagcgcacaagcagctgttattgatggtaccatagttggggctatcacttccattaacagtgaactgttaggtctaacacattggacaacaacacctaatttttattactactctatatataattacacaaatgataggactcgtggcactgcaattgacagtaatgatgttgattgtgaacctgtcataacctattctaacataggtgtttgtaaaaatggtgctttggtttttattaacgtcacacattctgatggagacgtgcaaccaattagcactggtaacgtcacgatacctacaaactttactatatccgtgcaagtcgaatatattcaggtttacactacaccagtgtcaatagactgtccaagatatgtttgtaatggcaaccctaggtgtaacaaattgttaacacaatacgtttctgcatgtcaaactattgagcaagcacttgcaatgggtgccagacttgaaaacatggaagttgattccatgttatttgtttctgaaaatgcccttaaattggcttctgtcgaagcatccaatagttcagaaactttagatcctatttacaaagaatggcctaatataggtggctcttggctagaaggtctaaaatacatacttccgtccgataatagcaaacgtaagtcagctatagaggacttgctttttgctaaggttgtaacgtctggtttaggtacagttgatgaagattataaacgttgtacaggtggttatgacatagctgacttagtatgtgctcaatactacaatggcatcatggtgctacctggtgtggctaatgctgacaaaatgactatgtacacagcatccctcgcaggtggtataacattaggtgcatttggtggaggcgccgtggctataccttttgcagtagcagttcaggctagacttaattatgttgctctacaaactgatgtattgaacaaaaaccagcagatcctggctagtgctttcaatcaagctattggtaacattacacagtcatttggtaaggttaatgatgcaatacatcaaacatcacgaggtcttgcaactgttgctaaagcattgccaaaagtgcaagatgttgtcaacacacaagggcaagctttaagccacctaacagtacaattgcaaaataatttccaagccattagtagttctattagtgacatttataataggcttgatgaattgagtgctgatgcacaagttgacaggctgatcacaggaagacttacagcacttaatgcatttgtgtctcagactctaaccagacaagccgaggttagggctagtagacaacttgccaaagacaaggttaatgaatgcgttaggtctcagtctcagagattcggattctgtggtaatggtacacatttgttttcactcgcaaatgcagcaccaaatggcatgatcttctttcacacagtgctattaccaacggcttatgaaactgtgactgcttgggcaggtatttgtgctttagatggtgatcgcacttttggacttgtcgttaaagatgtccagttgactttgtttcgtaatctagatgacaagttctatttgacccccagaactatgtatcagcctagagtggcaactagttctgattttgttcaaattgaagggtgcgatgtgctgtttgttaatgcaactgtaagtgatttgcctagtattatacctgattatattgatattaatcagactgttcaagacatattagaaaattttagaccaaattggactgtacctgagttgacatttgacatttttaacgcaacctatttaaacctgactggtgaaattgatgacttagaatttaggtcagaaaagctacataacactactgtagaacttgccattcttattgacaacattaacaatacattagtcaatcttgaatggctcaataggattgaaacctatgtaaaatggccttggtatgtgtggctactaataggcttagtagtaatattttgcataccattactgctattttgctgttgtagtacaggttgctgtggatgcataggttgtttaggaagttgttgtcactctatatgcagtagaagacgatttgaaaattacgaacctattgaaaaagtgcacgtccattaa

SEQ ID NO: 2 - protein, 1447 aa, Transmissible Gatroenteritis Virus Spike protein
MKKLFVVLVVMPLIYGDNFPCSKLTNRTIGNHWNLIETFLLNYSSRLSPNSDVVLGDYFPTVQPWFNCIRNNSNDLYVTLENLKALYWDYATENITSNHKQRLNVVVNGYPYSITVTTTRNFNSAEGAIICICKGSPPTTTTESSLTCNWGSECRLNHKFPICPSNSEANCGNMLYGLQWFADAVVAYLHGASYRISFENQWSGTVTLGDMRATTLETAGTLVDLWWFNPVYDVSYYRVNNKNGTTVVSNCTDQCASYVANVFTTQPGGFIPSDFSFNNWFLLTNSSTLVSGKLVTKQPLLVNCLWPVPSFEEAASTFCFEGAGFDQCNGAVLNNTVDVIRFNLNFTTNVQSGKGATVFSLNTTGGVTLEISCYNDTVSDSSFSSYGEMPSGVTDGPRYCYVLYNGTALKYLGTLPPIVKEIAISKWGHFYINGYNFFSTFPIDCISFNLTTGDSDVFWTIAYTSYTEALVQVENTAITKVTYCNSYVNNIKCSQLTANLNNGFYPVSSSEVGLVNKSVVLLPSFYTHTIVNITIGLGMKRSGYGQPIASTLSNITLPMQDNNTDVYCIRSDQFSVYVHSTCTSSLWDNVFKRNCTDVLDATAVIKTGTCPFSFDKLNNYLTFNKFCLSLSPVGANCKFDVAARTRTNDQVVRSLYVIYEEGDNIVGVPSDNSGLHDLSVLHLDSCTDYNIYGRTGVGIIRQTNRTLLSGLYYTSLSGDLLGFKNVSDGVIYSVTPCDVSAQAAVIDGTIVGAITSINSELLGLTHWTTTPNFYYYSIYNYTNDRTRGTAIDSNDVDCEPVITYSNIGVCKNGALVFINVTHSDGDVQPISTGNVTIPTNFTISVQVEYIQVYTTPVSIDCPRYVCNGNPRCNKLLTQYVSACQTIEQALAMGARLENMEVDSMLFVSENALKLASVEASNSSETLDPIYKEWPNIGGSWLEGLKYILPSDNSKRKSAIEDLLFAKVVTSGLGTVDEDYKRCTGGYDIADLVCAQYYNGIMVLPGVANADKMTMYTASLAGGITLGAFGGGAVAIPFAVAVQARLNYVALQTDVLNKNQQILASAFNQAIGNITQSFGKVNDAIHQTSRGLATVAKALPKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWAGICALDGDRTFGLVVKDVQLTLFRNLDDKFYLTPRTMYQPRVATSSDFVQIEGCDVLFVNATVSDLPSIIPDYIDINQTVQDILENFRPNWTVPELTFDIFNATYLNLTGEIDDLEFRSEKLHNTTVELAILIDNINNTLVNLEWLNRIETYVKWPWYVWLLIGLVVIFCIPLLLFCCCSTGCCGCIGCLGSCCHSICSRRRFENYEPIEKVHVH